(19) United States
(12) Patent Application Publication
(10) Pub. No.: US 2013/0066270 A1
(43) Pub. Date: Mar. 14, 2013

(54) DISPOSABLE SAFETY SELF-DESTROYED SYRINGE

(71) Applicants: Hsueh-Jung LU, Taoyuan County (TW); Chin-Shu Tsai, Taoyuan County (TW)

(72) Inventors: Hsueh-Jung LU, Taoyuan County (TW); Chin-Shu Tsai, Taoyuan County (TW)

(21) Appl. No.: 13/228,894

(22) Filed: Sep. 9, 2011

(51) Int. Cl. A61M 5/50

(52) U.S. Cl. 604/110

(57) ABSTRACT

A disposable safety self-destroyed syringe comprises a plunger and a syringe tube. An end of the plunger is sleeved with a piston, and an elastic protrusive column is coupled inside the piston with its front end being protruded to a distance from a top surface of the piston; the plunger is inserted into the syringe tube from its front end for injection, and the rear end of the syringe tube is contracted radially to form a needle seat having a blocking portion on the inner wall to be conjoined with a needle, so that when the plunger is depressed to reach closely to the rear end of the syringe tube, a front end of the protrusive column is inserted into the needle seat and is stopped by the blocking portion on the inner wall of the needle seat to retain the protrusive column inside the needle seat.

### BACKGROUND OF THE INVENTION

1. Field of Invention

The present invention relates to a disposable safety self-destroyed syringe and more particularly to disposable safety self-destroyed syringe which can effectively reduce pain of patients caused by the force applied in injections and residual amount of injection left in a syringe.

2. Related Art

Currently, injectors or syringes for hypodermic injections are widely applied in the medical field; generally, the injectors can only be used once and are disposed after being used, which are convenient and hygienic to avoid infection of diseases through needles. However, medical personnel can be pricked negligently by exposed needles of disposed injectors, to cause wounds or infected with diseases. If disposed injectors are not handled properly and are discarded in an open environment, others can be easily prinked by the sharp-pointed needles. Environmental workers who handle medical waste are potential victims of the disposed needles. Therefore, many types of safety injectors are produced.

Referring to FIG. 7, it shows a conventional safety syringe structure. A plunger “a” can be pushed or pulled forwards or backwards inside a syringe tube “b”. A front end of the plunger “a” has a piston “c” and a hook “d”. The hook “d” is disposed outside a front end surface of the piston “c”. A retaining portion “f” is disposed on a bottom of a needle “e”. When the plunger “a” is pushed to the end, the hook “d” is inserted into the retaining portion “f” to be conjoined together.

However, during the last stage of an injection, in order to have the piston “c” to be depressed to the end, a considerably force is used to push the plunger “a” so that the hook “d” can be conjoined together with the retaining portion “f”. The force can cause pain to patients. In addition, even though the piston “c” is depressed to the end, residual injection is left between the syringe tube “b” and the needle “e”. Therefore, medical personnel will have to draw more liquid medicament to avoid the medical effects being reduced because of the inadequate amount of medicament. Furthermore, the amount of medicament being drawn is inadequate because of negligence of the medical personnel, it will also reduce the medical effects and affect the patient's conditions.

### SUMMARY OF THE INVENTION

An object of the present invention is to provide a disposable safety self-destroyed syringe which can effectively reduce pain of patients caused by the force applied in injections.

Another object of the present invention is to provide a disposable safety self-destroyed syringe which can effectively reduce residual amount of injection left in a syringe.

In order to achieve the above-mentioned objects, the present invention of a disposable safety self-destroyed syringe comprises a plunger and a syringe tube. An end of the plunger is sleeved with a piston. A protrusive column, which is elastic, is coupled inside the piston. A front end of the protrusive column is protruded to a distance from a top surface of the piston. The syringe tube has a front end and a rear end, the plunger is inserted into the syringe tube from its front end for injection, while the rear end is contracted radially to form a needle seat to be conjoined with a needle. A blocking portion is disposed on an inner wall of the needle seat; so that when the plunger is depressed to reach closely to the rear end of the syringe tube, a front end of the protrusive column is inserted into the needle seat and is stopped by the blocking portion on the inner wall of the needle seat; therefore the protrusive column is retained inside the needle seat. At least one orifice is disposed on the protrusive column for buffering the force of inserting the front end of the protrusive column into the needle seat. The plunger can be detached from the piston and taken out when it is drawn back.

When it is embodied, a wing-shaped portion is disposed at a rear end of the protrusive column for conjoining inside the piston.

When it is embodied, the front end of the protrusive column is formed in a tapered shape.

When it is embodied, the front end of the protrusive column extends upward to form a long post.

When it is embodied, said piston and said protrusive column are made of elastic material.

The present invention will become more fully understood by reference to the following detailed description thereof when read in conjunction with the attached drawings.

### BRIEF DESCRIPTION OF THE DRAWINGS

FIG. 1 is a perspective view of a disposable safety self-destroyed syringe of a preferred embodiment of the present invention before assembly;

FIG. 2 is a perspective view of a disposable safety self-destroyed syringe of a preferred embodiment of the present invention after assembly;

FIG. 3 is an illustration of a disposable safety self-destroyed syringe of a preferred embodiment of the present invention with a front end of a protrusive column being inserted into a needle seat;

FIG. 4 is an illustration of a disposable safety self-destroyed syringe of a preferred embodiment of the present invention after a front end of a protrusive column is inserted into a needle seat;

FIG. 5 is an illustration of a disposable safety self-destroyed syringe of a preferred embodiment of the present invention after a plunger is detached from a piston and being taken out;

FIG. 6 is a perspective view of a protrusive column of a disposable safety self-destroyed syringe of another preferred embodiment of the present invention; and

FIG. 7 is an illustration of a safety syringe structure of a prior art.

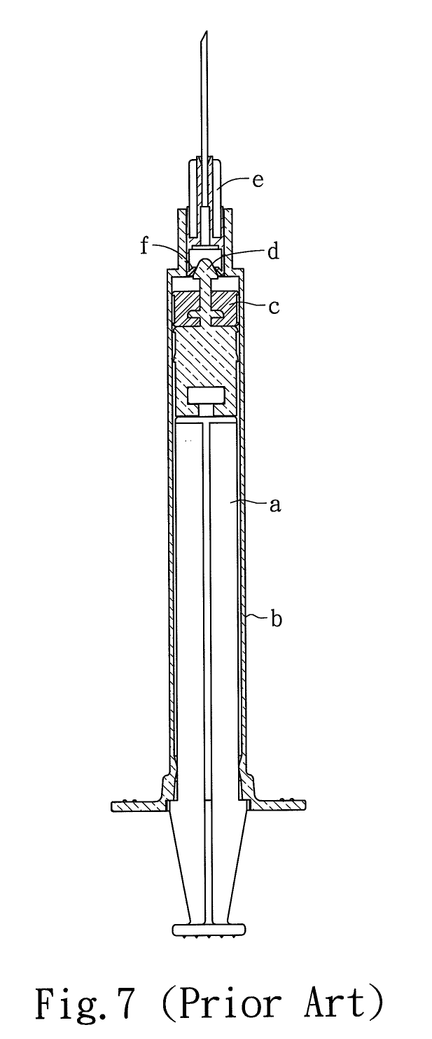

### DETAILED DESCRIPTION OF THE INVENTION

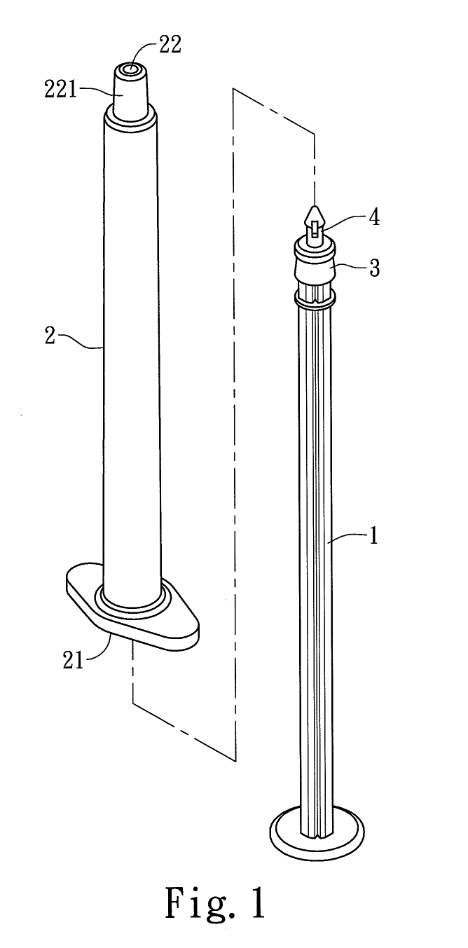

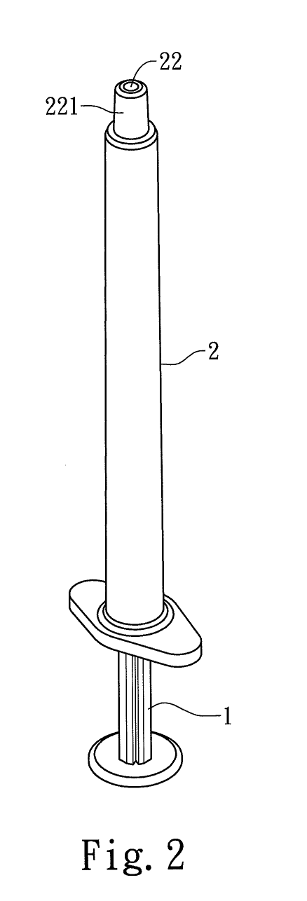

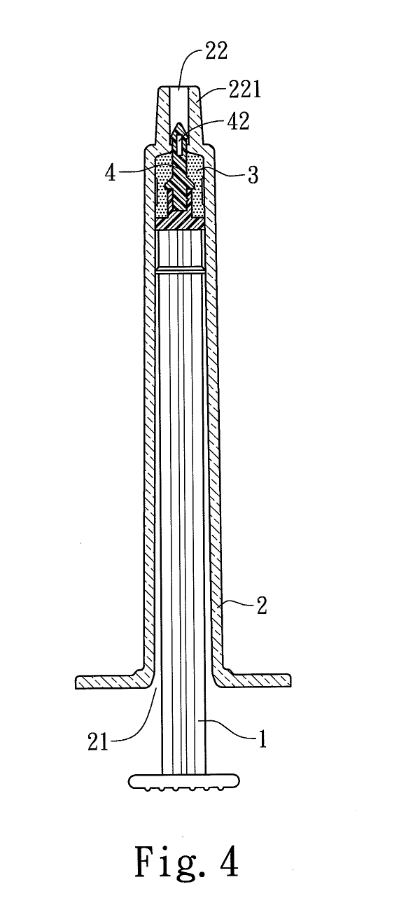

As shown in FIGS. 1 to 5, they refer to an embodiment of a disposable safety self-destroyed syringe of the present invention, which comprises a plunger 1 and a syringe tube 2.

An end of the plunger 1 is sleeved with a piston 3 which is made of elastic material. A protrusive column 4, which is also made of elastic material, is coupled inside the piston 3. A front end of the protrusive column 4 is in a tapered shape and is protruded to a distance from a top surface of the piston 3. Wherein, a wing-shaped portion 41 is disposed at a rear end of the protrusive column 4 for conjoining inside the piston 3. At least one orifice 42 is disposed on the protrusive column 4.

The syringe tube 2 has a front end 21 and a rear end 22, wherein the plunger 1 is inserted into the syringe tube 2 from its front end 21 for injection, while the rear end 22 is contracted radially to form a needle seat 221 to be conjoined with a needle. A blocking portion 222 is disposed on an inner wall of the needle seat 221.

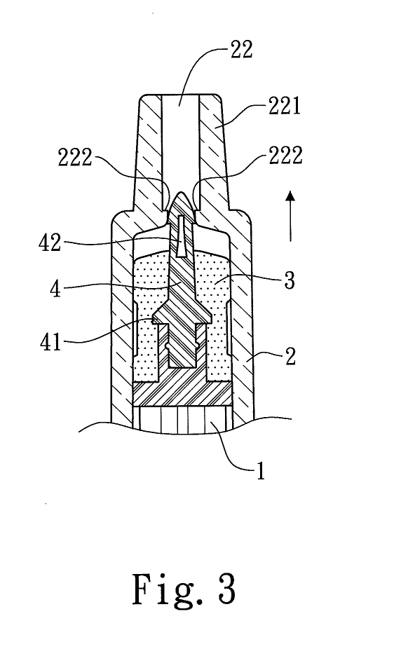

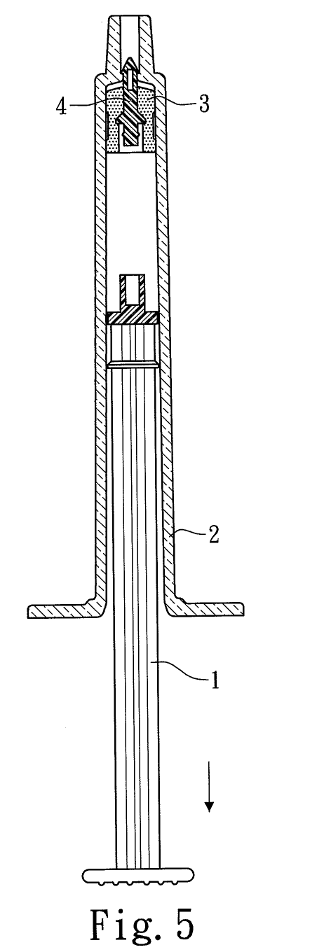

When it is embodied as shown in FIGS. 3 to 5, in a medicament injection, the plunger 1 is depressed to the rear end 22 of the syringe tube 2, so that the front end of the protrusive column 4 is inserted into the needle seat 221; it is then being stopped by the blocking portion 222 on the inner wall of the needle seat 221; consequently to be retained inside the needle seat 221. At the same time, when a force is exerted on the protrusive column 4; because at least one orifice 42 is disposed on the protrusive column 4, so that the protrusive column 4 is compressed to deform; in order to buffer the force of inserting the front end of the protrusive column 4 into the needle seat 221. When the injection is done and needs to be disposed; because the front end of the plunger 1 is retained inside the needle seat 221 and cannot be taken out, and also because of the sleeved conjunction design between the plunger 1 and the piston 3; by pulling the plunger 1, it can be easily detached from the piston 3 (shown in FIG. 5) and taken out. The syringe tube 2 with the needle together can then be destroyed.

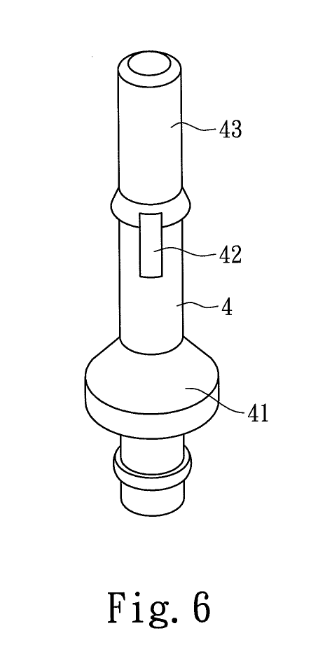

Moreover, as shown in FIG. 6, the front end of the protrusive column 4 extends upward to form a long post 43; therefore, when the protrusive column 4 is depressed to reach closely to the rear end of the syringe tube 2, the long post 42 at the front end of the protrusive column 4 can be inserted further inside the needle seat 221; so as to reduce the residual amount of injection.

Therefore, a disposable safety self-destroyed syringe of the present invention has the following advantages:
1. In the present invention, at least one orifice is disposed on the protrusive column, so that the protrusive column can be compressed to deform, in order to buffer the force of inserting the front end of the protrusive column into the needle seat, and therefore can effectively reduce pain of patients because of the exerted force.
2. In the present invention, the front end of the protrusive column extends upward to form a long post, therefore, it can be inserted further inside the needle seat, so as to reduce the residual amount of injection.
3. After a disposable safety self-destroyed syringe of the present invention is used, the plunger can be pulled to detach from the piston and then taken out from the syringe tube. It can be reclaimed and reprocessed for future applications and therefore can effectively reduce both waste and costs.

According to the above disclosure, the present invention can certainly achieve the expected objects by providing a disposable safety self-destroyed syringe which can be practically applied in related industries.

Although the embodiments of the present invention have been described in detail, many modifications and variations may be made by those skilled in the art from the teachings disclosed hereinabove. Therefore, it should be understood that any modification and variation equivalent to the spirit of the present invention be regarded to fall into the scope defined by the appended claims.

What is claimed is:

1. A disposable safety self-destroyed syringe comprising:
a plunger with one end sleeved with a piston, wherein an elastic protrusive column is coupled inside said piston, and a front end of said protrusive column is protruded to be in a distance away from a top surface of said piston;
a syringe tube including a front end and a rear end, wherein said plunger is inserted into said syringe tube from said front end for injection, while said rear end is contracted radially to form a needle seat to be connected with a needle; a blocking portion is disposed on an inner wall of said needle seat, so that when said plunger is pushed to reach closely to said rear end of said syringe tube; said front end of said protrusive column is inserted into said needle seat and stopped by said blocking portion on said inner wall of said needle seat, so that said protrusive column is retained inside said needle seat, and said plunger can be detached from said piston and taken out when being drawn back; the improvement of said disposable safety self-destroyed syringe is that:
at least one orifice is disposed on said protrusive column, so as to buffer the force of inserting said front end of said protrusive column into said needle seat.

2. The disposable safety self-destroyed syringe as claimed in claim 1, wherein a wing-shaped portion is disposed at a rear end of said protrusive column to be engaged into said piston.

3. The disposable safety self-destroyed syringe as claimed in claim 1, wherein said front end of said protrusive column is in a tapered shape.

4. The disposable safety self-destroyed syringe as claimed in claim 3, wherein said front end of said protrusive column extends upward to form a long post.

5. The disposable safety self-destroyed syringe as claimed in claim 1, wherein said piston and said protrusive column are made of elastic material.

6. The disposable safety self-destroyed syringe as claimed in claim 1, wherein said front end of said protrusive column extends upward to form a long post.
